(19) United States
(12) Patent Application Publication
(10) Pub. No.: US 2020/0034567 A1
(43) Pub. Date: Jan. 30, 2020

(54) DATA COMMUNICATION NETWORK

(71) Applicant: 1929803 Ontario Corporation (d/b/a Flosonics Medical), Sudbury (CA)

(72) Inventor: Iouri Besperstov, Innisfil (CA)

(21) Appl. No.: 16/526,929

(22) Filed: Jul. 30, 2019

Related U.S. Application Data
(60) Provisional application No. 62/711,796, filed on Jul. 30, 2018.

(51) Int. Cl. G06F 21/62; G16H 10/60; G16H 50/20; H04L 29/08

(52) U.S. Cl. CPC G06F 21/6254; H04L 67/12; G16H 50/20; G16H 10/60

(57) ABSTRACT

A data communication network with a diagnostic module that collects biomedical patient data from a patient and wirelessly transmits a data packet comprising a unique identifier associated with the patient and at least a portion of the biomedical patient data. The unique identifier and the biomedical patient data in the data packet contains no private personal information about the patient's identity. A mobile device wirelessly receives the data packet from the diagnostic module and wirelessly transmit the data packet to a cloud-base server. The cloud-based server has a database that correlates the patient data via the unique identifier with the patient and stores the data packet. The cloud-based server provides some or all of the patient data in the data packet to an authorized recipient via a client data terminal, which correlates the unique identifier to patient information at the client data terminal.

### CROSS-REFERENCE TO RELATED APPLICATION(S)

This non-provisional patent application claims the benefit of and priority to U.S. Provisional Patent Application No. 62/711,796, titled “DATA COMMUNICATION NETWORK” and filed Jul. 30, 2018, which is incorporated herein by reference in its entirety.

### TECHNICAL FIELD

The present application relates to data communication networks.

### BACKGROUND

The transfer of data between parties has become ubiquitous. The transfer is accomplished between electronic communication devices connected to one another in a network. The devices will communicate with one another according to one or more predefined protocols that enable the recipient to receive and process a message sent by a sender according to the defined protocol.

As the use of networks has increased, the need for security and privacy has also increased to avoid dissemination of sensitive information and corruption of the information. As a result, the protocols used will incorporate measures that secure the transmission, usually by encryption of the message. This adds overhead to the device generating the message and the device receiving the message, requiring greater processing capability and increasing the bandwidth of network.

As the use of data networks continues to expand, as in the incorporation of a multitude of devices in the “internet of things”, the burdens placed on the networks escalates. A particular environment where the need for rapid and reliable exchange of information is essential is in monitoring biomedical devices, such as might be used in medical facilities or similar environments.

The need for privacy is paramount, but the conventional way of securing the data leads to a relatively complex, high bandwidth network.

It is therefore an object of the present technology to obviate or mitigate the above disadvantages.

### SUMMARY

The data communication network technology of the present disclosure overcomes drawbacks of the present disclosure and provides additional benefits. In one embodiment of the technology, a system is configured to wirelessly and anonymously transmit biomedical patient data to and from a cloud-based server. The system comprises a diagnostic module configured to collect biomedical patient data from a patient and wirelessly transmit a data packet comprising a unique identifier associated with the patient and at least a portion of the biomedical patient data. The unique identifier and the biomedical patient data in the data packet contain no private personal information about the patient's identity. A mobile device is configured to wirelessly receive the data packet from the diagnostic module and wirelessly transmit the data packet to a cloud-base server. The cloud-based server has a database and is configured to correlate the patient data via the unique identifier with the patient and to store the data packet in the database. The cloud-based server is configured to provide some or all of the data packet to an authorized recipient via a client data terminal configured to correlate the unique identifier to patient information at the client data terminal.

Another embodiment of the present technology provides a method for wirelessly collecting and saving biomedical patient data. The method includes defining a de-personalized unique identifier for a patient by receiving, at a cloud-based server, patient identification data transmitted from a client data terminal, and generating a unique identifier associated with the patient identification data at the server. The patient identification data includes private personal information about the patient and the patient's identity. The unique identifier contains no private personal information about the patient and the patient's identity. The method includes associating a diagnostic module with the unique identifier and providing the unique identifier from the server to the diagnostic module. The diagnostic module is configured to collect biomedical patient data from a patient and to wirelessly transmit a data packet comprising the unique identifier and at least a portion of the real-time biomedical patient data. The data packet contains no private personal information about the patient's identity. The cloud-based server receives the data packet sent from the diagnostic module and is configured to provide some or all of the data packet to an authorized recipient via a client data terminal configured to correlate the unique identifier to patient information at the client data terminal.

Another embodiment provides a method for wirelessly transmitting biomedical patient data. The method includes receiving at a cloud-based server a data packet comprising a unique identifier associated with a patient and at least a portion of biomedical patient data collected in real time from the patient. The unique identifier includes no private personal information about the patient or the patient's identity. The data packet is wirelessly received from a mobile device or a router that has received the portion of the biomedical patient data and the unique identifier associated with the patient from a diagnostic module. The cloud-based server authenticates a client data terminal and wirelessly transmits to the client data terminal the data comprising the unique identifier and at least a portion of the biomedical patient data. The data wirelessly transmitted to the client data terminal contains no private personal information about the patient's identity.

### BRIEF DESCRIPTION OF THE DRAWINGS

FIG. 1 is a schematic representation of a network to transfer patient data in accordance with at least one embodiment of the disclosed technology.

FIG. 2 is a flow chart illustrating the enrollment of a patient and the monitoring devices associated with the patient within the network of FIG. 1.

FIG. 3 is a flow chart of the uploading or transmitting of biomedical patient data from the wireless diagnostic module to the cloud-based server in accordance with at least one embodiment of the disclosed technology.

FIG. 4 is a flow chart illustrating transferring and storing data when an alarm condition is detected by the wireless diagnostic module in accordance with at least one embodiment of the disclosed technology.

The systems, components and/or techniques introduced herein may be better understood by referring to the following Detailed Description in conjunction with the accompanying drawings, in which like reference numerals indicate identical or functionally similar elements.

### DETAILED DESCRIPTION

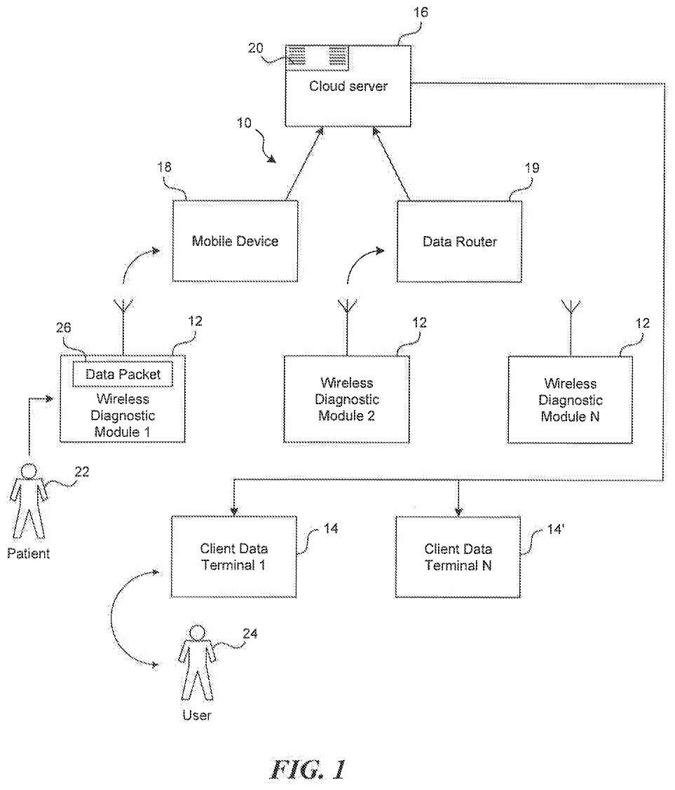

FIG. 1 is a schematic representation of a data communication network 10 configured to receive and transfer anonymous patient-related data to protect patient identity. In general, the network 10 communicates data collected from one or more wireless diagnostic modules 12 through a mobile device 18 (and/or a data router 19) and a cloud-based server 16 to one or more client data terminals 14.

The wireless diagnostic module 12 collects data associated with different biomedical parameters for a patient 22 and stores the parameters for a limited period. The data collected by the wireless diagnostic module 12 will vary from device to device, and may be a single parameter, such as pulse, or combined parameters such as pulse and blood pressure and body temperature, each of which has a designated code to indicate the parameter sensed. Other biomedical, biological or diagnostic data, such as ECG, blood flow information, and the like may be detected. The value of the parameter sensed is stored as a numerical value. The parameter's numerical value is combined with the designated code and a unique identifier associated with the patient 22, as further discussed below, for transfer as data to the server 16.

In one embodiment, the wireless diagnostic module 12 may be an ultrasound patch for detecting fluid flow, which may be referred to herein as a Flopatch™, as disclosed in U.S. patent application Ser. No. 16/377,028, filed Apr. 5, 2019, which is incorporated herein by reference. In other implementations, the wireless diagnostic module 12 may be an EKG monitor, a pulse oximeter, temperature sensor, movement sensor, blood pressure sensor, or other sensor not specifically named herein that is used to detect biomedical patient data.

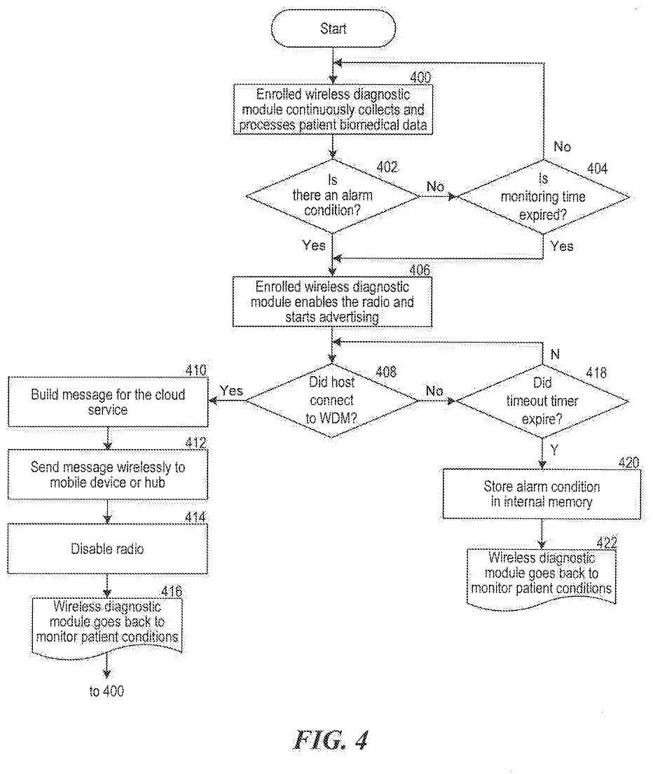

The wireless diagnostic module 12 may also compare the collected patient data to preset parameters or limits. If the patient data exceeds a limit, the wireless diagnostic module 12 may generate an alarm or alert, as discussed further below in FIG. 4.

The wireless diagnostic module 12 transmits the stored data to the mobile device 18 or router 19. The wireless diagnostic module 12 may include a Bluetooth Low Energy (BLE) communication function that permits communication of the diagnostic device 12 with the mobile device 18 and/or server 16. Other wireless communication formats and methods may be used.

The mobile device 18 transmits the data to the server 16 without altering the data. Alternatively, the wireless diagnostic module 12 may transmit the stored data to the server 16 directly. The client data terminal 14 can then access the patient data from the server 16, or the server 16 may be configured to transmit or push the patient data to one or more client data terminals 14 at predetermined intervals.

In the illustrated embodiment, the wireless diagnostic device 12 has different states. A “Released Device” is a diagnostic device 12 that has not been associated with a patient. As a Released Device, the wireless diagnostic module 12 can perform measurements on demand when instructed by a mobile device 18. An “Enrolled Device” is a device that has been associated with a patient. The Enrolled Device is configured to acquire biomedical patient data, process the biomedical patient data, and to transfer one or more data packets 26 that include data based on the biomedical patient data to the server 16 via mobile device 18 or data router 19 within a certain period of time or over predetermined time intervals.

The mobile device 18 may be a smart phone, tablet, or similar computing device running an application programming interface (API) or is a data router 19, or data router hub, that transfers data received to the server 16. Other devices and applications may be used. The mobile device 18 and router 19 accept the data and transmit the data to the cloud-based server 16. The mobile device 18 and router 19 may have no personally identifiable information about the patient 22. In some embodiments, the mobile device 18 and router act only as data routers. In other embodiments, a device, such as a portable personal electronic device can be configured in one mode or program (i.e., software or application running on the device) to operate as the mobile device 18 and operate in another mode or program as the client data terminal 14 to view patient data that has been stored in the database 20.

The mobile device 18 running the API may perform at least the functions of data routing, client and module 12 configuration or enrollment, and patient data review. For data routing, when the API is running on a mobile device 18 and senses a nearby active, enrolled wireless diagnostic module 12 trying to send data, the API automatically connects to the module 12 and routes data to the cloud server 16. The data transfer requires no action by the owner of the mobile device 18 to transmit the data, such that the data transfer may occur automatically without the owner's knowledge. The owner may have no direct access via the mobile device 18 to the data being transferred to the cloud. For enrollment, in this mode and after system authentication, a patient 22 can be added to the database 20 and a wireless diagnostic module 12 can be associated with the patient 22. For patient data review, an authenticated “Protected Entity” is allowed to review individually identifiable patient data.

The cloud-based server 16 receives data from the mobile device 18 and router 19 and performs multiple tasks such as those discussed herein. The tasks are not limited, and other embodiments are contemplated. The server 16 also maintains a database 20 that correlates a unique identifier (UID) with an enrolled patient 22.

The cloud-based server 16 may authenticate a user 24, thus allowing the user 24 to see data of one or more patients 22 via the client data terminal 14. The cloud-based server 16 may encrypt transmitted patient data and accomplish data storage, such as storing large data files from a patient 22 such as ECG/EMG/EEG and the like. The real-time database 20 is configured to perform functions such as: maintaining an authenticated association link between the patient 22 and the wireless diagnostic module 12; maintaining an authenticated association between the user 24 and patient(s) 22 for whom the user 24 is authorized to view data; store small dynamic data such as pulse/temperature/blood pressure: and store patient personal information, such as a patient's hospital records. The cloud-based server 16 can also be configured to generate alerts. Alerts are discussed in more detail with respect to FIG. 4. Alerts advise if any preset physiological parameters are exceeded and if a potential critical condition exists based on data from a wireless diagnostic module 12. This function can send an alert to the client data terminal for notification of the user(s) 24, such as authorized caregivers, parents, relatives, and nursing staff via SMS, e-mail, application notification, or other method of notification.

Each client data terminal 14 may be associated with a specific authorized user, such as a medical practitioner in the medical environment, or a team of users, such as a group of medical practitioners. Accordingly, the data collected is available to the single or group of authorized users. In some implementations, one or more wireless diagnostic modules 12 may wirelessly transmit data to the cloud-based server 16 directly. In other implementations, patient data may be viewed on the mobile device 18.

In some implementations, the devices of network 10 may include a communication device capable of communicating wirelessly or may communicate wire-based with a network node. Some devices may be capable of both wireless and wire-based communication. The communication device can communicate with another device or a server through a network using, for example, TCP/IP protocols, a Q-LAN protocol, or others. Operations may be distributed across multiple network devices as needed to transmit requests and patient data as some devices may not always be available to the network 10.

In some implementations, the devices of network 10 may have access to a memory that is within the device or distributed across multiple devices. A memory includes one or more of various hardware devices for volatile and non-volatile storage and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers and so forth. A memory is not a propagating signal divorced from underlying hardware; a memory is thus non-transitory.

The client data terminal 14 may be a mobile device or a hard-wired device running an API or a Webpage generated by the cloud-based server 16 individually for each client data terminal 14. The client data terminal 14 allows authenticated users to review stored data as well as current results and diagnosis. The client data terminal 14 can include at least one output for presenting the patient data, such as a display, a printer, a speaker, and the like.

There may be multiple client data terminals 14. For example, one client data terminal 14′ may be a computer connected to the internet. The client data terminal 14′ may be located remote from the wireless diagnostic module 12, for example, in a different hospital, clinic, a residence, or a different geographic region.

The function of the network 10 will be discussed below with respect to the four modules 12, 14, 16, 18. The wireless diagnostic module 12 collects biomedical patient data from the patient 22. The module 12 may provide some processing and/or preprocessing of the patient data. The module 12 associates the numerical value of the parameter with an appropriate designation code and assembles a data string that is a concatenation of the designation code and the numerical value. The data string may include other information, although the information contains no patient-identifying information except for a general, anonymous UID associated with the patient during registration or enrollment.

The designation codes may be based on an agreed set of designations applicable to all devices, such as 01 indicating pulse, 02 indicating temperature, and the like. Alternatively, a designation code may be mapped to each wireless diagnostic module 12 individually at registration. In the latter case, a cross-correlation table is provided to the server 16, so each module's 12 designation may be mapped into a general set of parameters on the database 16.

Access to the data managed by the server 16 may be tailored to the demands of the particular environment. In the context of a medical device environment, a patient 22 is a subject who wears or is connected to a wireless diagnostic module 12 but has no access authorization to the database 20 unless authorized.

Access to data collected by the wireless diagnostic module 12 is controlled to one or more users 24 who are recognized as “Protected Entities.” Each user 24 may have differing access according to the authority accorded to the user 24. For example, a “Caregiver” may be a Protected Entity who is allowed to access biomedical information of one or more assigned patients. The Caregiver may view information on the client data terminal 14. A “Medical professional” may be a Protected Entity who is allowed to access data from multiple patients, manage patient information, and authorize other Protected Entities or other specified individuals to access data. The Medical Professional may view information on the client data terminal 14. A “Cloud Administrator” may be a Protected Entity who monitors the activity, the users and the functionality of the Cloud Service and network 10. The Cloud Administrator has rights to manage and control the access of all the users 24.

Some implementations can be operational with numerous other computing system environments or configurations. Examples of computing systems, environments, and/or configurations that may be suitable for use with the technology include, but are not limited to, personal computers, networked peripherals, video conference consoles, server computers, handheld or laptop devices, cellular telephones, wearable electronics, gaming consoles, tablet devices, multiprocessor systems, microprocessor-based systems, set-top boxes, programmable consumer electronics, network PCs, minicomputers, mainframe computers, distributed computing environments that include any of the above systems or devices, or the like.

Those skilled in the art will appreciate that the components illustrated in FIG. 1 described above, and in each of the flow diagrams discussed below, may be altered in a variety of ways. For example, the order of the logic may be rearranged, substeps may be performed in parallel, illustrated logic may be omitted, other logic may be included, etc. In some implementations, one or more of the components described above can execute one or more of the processes described below.

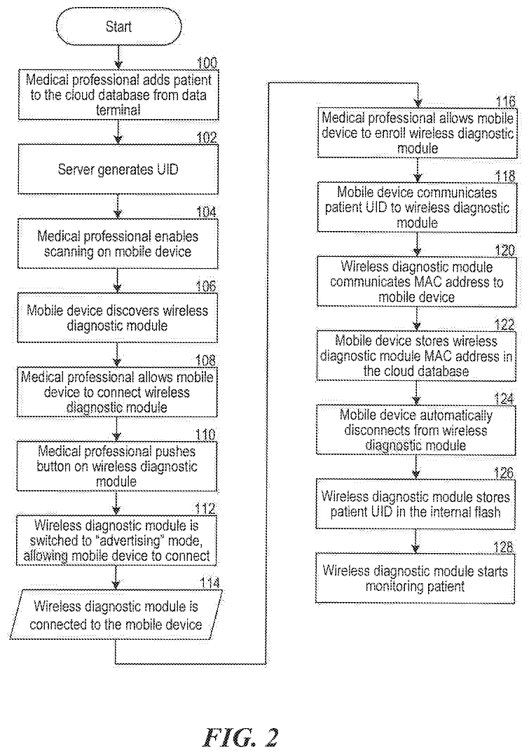

FIG. 2 is a flow chart illustrating the enrollment of the patient 22 and the wireless diagnostic modules 12 associated with the patient 22 within the network 10 of FIG. 1. At step 100, an eligible user 24, such as the Medical Professional or Cloud Administrator, logs into the API on the client data terminal 14. Login protocols, such as an email or other electronic invitation and established credentials, may be used. Once logged in, the user 24 can add a patient 22 to the cloud database 20. Personal data associated with the patient 22 may be added, such as full patient name, date of birth, photo identification, diagnosis, symptoms, Hospital Record Number (HRN), and the like. Alternatively, the user 24 may log into a program, App, or other portion of the mobile device 18 and add the patient 22 to the cloud database 20.

At step 102 the server 16 or cloud database 20 will generate an anonymous UID for the patient 22 (i.e., the UID is anonymous to any unauthorized third party). Any further communications between components of the network 10 will use this UID rather than patient personal information. With the patient 22 enrolled and the UID issued, the user 24 associates a wireless diagnostic module 12 with the patient's UID, so the UID is stored in the wireless diagnostic module's memory, such as the flash memory or other associated memory.

At step 104 the user 24 enables or activates the mobile device 18 to initiate a scanning mode to locate the selected wireless diagnostic module 12. At step 106 the mobile device 18 wirelessly detects the identified transmitting wireless diagnostic module 12. At step 108 the user 24 allows the mobile device 18 to utilize the wireless communication, such as BLE communication, to connect to the wireless diagnostic module 12. At step 110 the user 24 activates a registration button on the wireless diagnostic module 12. At step 112 the wireless diagnostic module 12 switches to an “advertising” mode, which allows the mobile device 18 to connect to the wireless diagnostic module 12. At step 114 at the module 12 and the device 18 are connected or linked. At step 116 the user 24 allows the device 18 to enroll the module 12 with the cloud server 16. At step 118 the mobile device 18 obtains the IAD from the database 20 and communicates the UID to the module 12. At step 120 the wireless diagnostic module 12 provides the module's unique module identifier, such as a media access control address (MAC), to the device 18. At step 122 the mobile device 18 transfers the MAC to the database 20 for correlation with the UID.

If multiple wireless diagnostic modules 12 are associated with a patient 22, for example to collect a wider range of data, the MAC address of each of the wireless diagnostic modules 1-N 12 will be associated with the same UID so as to link the modules 12 to a common patient 22. At step 124 the mobile device 18 disconnects from the module 12. At step 126 the wireless diagnostic module 12 stores the UID in its internal flash memory. At step 128 the wireless diagnostic module 12 now begins to monitor patient 22 and collect real-time biomedical patient data. It will be appreciated that during connection, registration and enrollment, the mobile device 18 is functioning with the API and does not store data within the mobile device 18.

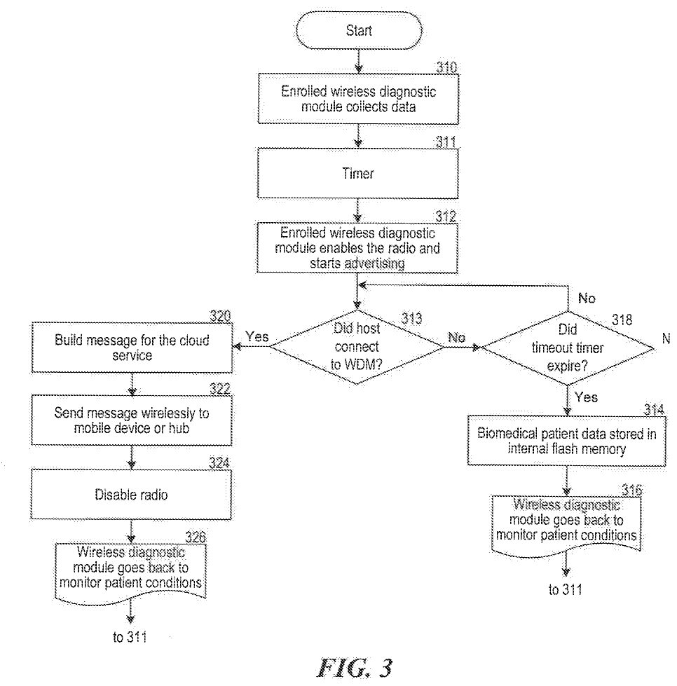

FIG. 3 is a flow chart of monitoring of biomedical patient data and the uploading or transmitting of biomedical patient data from the wireless diagnostic module 12 to the cloud-based server 16. At step 310 the wireless diagnostic module 12 constantly or regularly collects and processes de-identified biomedical client data. In another embodiment, the biomedical data may be acquired at preset intervals. At step 311, a timer runs for one or more designated intervals. In one embodiment, the timer runs for five minutes.

After the designated interval, at step 312 the wireless diagnostic module 12 activates an internal or integrated radio or transmitter and tries to connect to a mobile device 18 or router 19 running the API. This may be any authorized mobile device 18 that is running the API that is in the proximity of the wireless diagnostic device 12, whether or not the owner of the mobile device 18 is in any way associated with the patient 22.

If, at step 313 the mobile device 18 did not connect to the wireless diagnostic device 12 within a predetermined amount of time, at step 318 the wireless diagnostic device 12 determines whether a timeout timer has expired. In one embodiment, a timeout timer is used so that the wireless diagnostic device 12 conserves power by not continually attempting to transmit data when no mobile device 18 is available. If the timeout timer has not expired, the process returns to step 313.

If the timeout timer has expired, the process passes to step 314 where the wireless diagnostic module 12 stores the biomedical client data in local flash memory to be transferred on the next successful connection to a device 18 running the API.

At step 316 the wireless diagnostic module 12 continues to sample real-time biomedical client data. It should be understood that the module 12 may continuously sample biomedical client data, storing the data locally. The process may then return to step 311.

Returning to step 313, if a mobile device 18 is located and a connection is successful, process proceeds to step 320. In general, the de-identified biomedical data is transferred from the module 12 to the device 18, and the device 18 transfers the data to the cloud server 16. The biomedical data uses only the UID so that the data is de-identified (i.e., does not include the patient's personal information about the patient's identity). Accordingly, the data transmitted from the module 12 with the UID and the sensed biomedical patient data has no private information regarding the patient's identity, so the transmitted data need not be encrypted. Similarly, the data received and transmitted by the mobile device 18 or router also need not be encrypted. Nonetheless, in some embodiments, the de-identified data may still be encrypted if desired.

The wireless diagnostic module 12 is connected or linked to the device 18 and the device 18 is authenticated through the cloud server 16. During this process, the cloud server 16 actively processes and stores the received biomedical data to the database 20 using the UID. The wireless diagnostic module 12 communicates with the server 16 through the mobile device 18 using its own unique module identifier (e.g., a MAC address), which the server 16 had correlated with the UID during enrollment of the diagnostic module. This correlation between the server 16 and the wireless diagnostic module 12 allows for the data to be properly stored in the server 16.

To upload data, as shown in FIG. 3, after a connection is established, at step 320 the wireless diagnostic module 12 builds a message or data packet 26 for the cloud server 16, which includes the UID associated with the patient 22 and biomedical data such as heart rate, SpO2 level, step counter, number of beats per minute, blood flow rate, percentage of the oxygen level, number of steps, temperature values (Celsius or Fahrenheit). Other data may be collected for transmission. The data packet 26 will contain the relevant data required for the recorded measurements, including a timestamp, but the data packet will typically use less than all of the data collected by the wireless diagnostic module 12. Similarly, the data packet 26 from one wireless diagnostic module 12 (e.g., a Flowpatch™) will have a standardized format consistent with data packet formats from other types of wireless diagnostic modules 12 (e.g., an EEG/EKG machine), which allows for improved data handling by the cloud server 16 and the client data terminals 14.

The message of the illustrated embodiment will typically have the form

wherein “ID” indicates the designation code and “Value” is an actual value of the measured parameter.

Once the data packet 26 is built, at step 322 the wireless diagnostic module 12 wirelessly transmits the message or data packet 26 to the mobile device 18 or data router 19. For example, the data may be transferred or transmitted via a BLE Link. Alternatively, when the mobile device 18 connects to the wireless diagnostic module 12, the mobile device 18 may extract the UID and individual biomedical parameters and push the data to the cloud. In another implementation, the data router 19 or hub may route de-identified data to the cloud server 16 through WIFI, cellular or Ethernet. The data may be extracted from the wireless diagnostic module 12 by the router 19 or pushed to the router 19.

The mobile device 18 or data router 19 then wirelessly transmits the message or data packet 26 to server 16. If mobile device 18 or router 19 has previously not been available, the wireless diagnostic module 12 will build and transmit a message or data packet 26 for each sample of de-identified patient biomedical data stored in internal memory of the wireless diagnostic module 12 once the link to the server 16 is established. Each message or data packet 26 is stored in the database 20 and correlated with the patient details and UID and MAC so a complete data set is obtained. At step 324 the wireless diagnostic module 12 disables the radio, and at step 326 the wireless diagnostic module 12 continues to acquire biomedical patient data, returning to step 311.

FIG. 4 is a flow chart illustrating the transfer and storage of data and storing data when an alarm condition is detected by the wireless diagnostic module 12. At step 400 the wireless diagnostic module 12 constantly monitors biomedical signals from the patient. At step 402 the wireless diagnostic module 12 determines whether an alarm condition has been met. Examples of alarm conditions may be high temperature, low or high pulse rate, lack of motion activities for a prolonged period of time, change of blood flow rate, or other condition state relative to a determined threshold. Other conditions may be monitored and be set to generate an alarm when exceeding a limit or failing to reach a predetermined limit.

If no alarm condition is detected, the process passes to 404, where the wireless diagnostic module 12 determines whether the monitoring timer has expired. If the monitoring timer has not expired, the process returns to step 400 and patient monitoring continues. If an alarm condition is detected at 402, the process passes to step 406, bypassing the timer to attempt to report those patient conditions to the cloud-based server 16 immediately. At step 406 the wireless diagnostic module 12 enables the radio/transmitter and enables the advertising mode, which allows the mobile device 18 to connect to the wireless diagnostic module 12. Therefore, the wireless diagnostic module 12 immediately attempts to connect to the mobile device 18 and/or data router 19 to communicate an alarm condition to the cloud server 16.

At step 408, if a connection is established between the wireless diagnostic module 12 and the mobile device 18 or data router 19, the process passes to step 410. At step 410 the wireless diagnostic module 12 builds a message or data packet 26 that includes at least the UID and the Type of Alarm. The Type of Alarm may be a number that is cross-referenced in a table stored at the database 20. Other alarm identifiers may be used. At step 412 the wireless diagnostic module 12 transfers the data packet 26 with the alarm condition the mobile device 18 and/or data router 19 and on to the cloud-based server 16. At step 414 the wireless diagnostic module 12 disables the radio/transmitter, and at step 416 the wireless diagnostic module 12 returns to monitoring patient conditions. The process returns to step 400.

If a connection is not established at step 408, at step 418 the wireless diagnostic module 12 determines whether the timeout timer has expired. If the timeout timer has not expired, the process returns to step 408 and the wireless diagnostic module 12 continues to attempt to connect to the mobile device 18 and data router 19. If the timeout timer at step 418 expires, the process passes to step 420 where the alarm condition is stored in internal memory of the wireless diagnostic module 12. The stored alarm condition(s) will be transferred to the could-based server 16 once a successful connection is made. At 422 the wireless diagnostic module 12 continues to monitor patient conditions and the process returns to step 400.

The data communication protocol between the wireless diagnostic module 12 and the mobile device 18 or data router 19 (which may also be referred to as a hub) is used to avoid unauthenticated connection between the wireless diagnostic module 12 and mobile device 18 or data router 19. When the wireless diagnostic module 12 starts advertising, selected custom parameters will be included in the advertising package. For example, the advertising package may include a Company Vender Unique Number, a Product Identifier (ID) and Enrollment Status of the wireless diagnostic module 12. Other parameters may be used. Some parameters, such as the Company Vendor Unique Number and the Product ID may be programmed in the memory of the Wireless Diagnostic Module 12 during the production and/or installation. A parameter, such as Enrollment Status, may be programmed in the wireless diagnostic module 12 during the enrollment process within the network 10. In one implementation, the Enrollment Status may be the UlD.

Once a Mobile Device 18 or data router 19 is scanning for a wireless diagnostic module 12, the mobile device 18 or data router 19 will be looking for those three parameters in an advertising package and will connect to the wireless diagnostic module 12 only if those three parameters are present in the advertising package. Other communication protocols may be used to prevent unauthenticated connection between the wireless diagnostic module and the mobile device 18.

After connection between the mobile device 18 or router 19 and the wireless diagnostic module 12 is established, the mobile device 18 or router 19 sends a handshake to the wireless diagnostic module 12. After receiving a handshake message from the mobile device 18 or router 19, the wireless diagnostic module 12 will prepare a message or data packet 26 as described above to include the UID and the value of the biomedical patient data. Once transmission between the wireless diagnostic module 12 and the mobile device 18 or router 19 is complete, the wireless diagnostic module 12 will disconnect from the mobile device 18 or router 19 and disable the radio/transmitter. The wireless diagnostic module 12 will go back to monitor patient biomedical data. The process of the mobile device 18 or router 19 wirelessly connecting to the diagnostic modules 12 using Bluetooth or other wireless protocols when the modules 12 have the de-identified data ready to transmit, and then disconnecting upon completion of the transmission, rather than continuing to maintain a wireless connection, allows the mobile device 18 and/or server 19 to communicate with a large number of wireless diagnostic modules 12 over a selected time period. This avoids some of the limitations of conventional wireless protocols that restrict the number of units to which a device can be continuously connected.

As discussed above, the wireless diagnostic module 12 performs single measurements and sends data to the cloud-based server 16. The biomedical patient data is then stored in the database 20. The following wireless diagnostic module streaming data format protocol may be used when larger amounts of streaming data are required or desired from the wireless diagnostic module 12. Examples of streaming data may be a doctor or other medical personnel requesting the display of heart wave/SpO2 wave from a pulse oximeter, identify blood flow rate from the Flopatch™, or any of electrocardiogram (ECG or EKG), electromyography (EMG), EDG, EEC, and the like.

Once connection between the wireless diagnostic module 12 and the mobile device 18 or router 19 is established, the wireless diagnostic module 12 will stream data to the mobile device 18 or router 19 according to the format below:

| | | | | |
|---|---|---|---|---|
| Patient | ID1 | Value 1.1 | Value 1.2 | Value 1.n |
| Cloud data | | | | |
| Patient | ID2 | Value 2.1 | Value 2.2 | Value 2.n |
| Cloud data | | | | |

In this format, the ID indicates the method or format used to measure values. For example, the ID may indicate an ECG 1-lead, ECG AvR lead, Pulse Oximetry IR-channel, Pulse Oximetry Red channel. Others may be used. The listed Values can be single samples or measurements, which may be concatenated to provide streaming data.

Once the mobile device 18 or router 19 receives the data packet 26 from the wireless diagnostic module 12, the mobile device 18 or router 19 does not send data immediately to the cloud-based server 16. Instead, the mobile device 18 or router 19 waits until measurements are complete and creates a comma separated value (CSV) file with data points and then uploads the CSV file to the cloud-based server 16 rather than sending many data packets 26 of individual data points. Other formats may be used to collect the individual data points into a file.

In some embodiments, the data router 19 is a passive device that connects to the wireless diagnostic module 12 if several conditions are met. For example, the router 19 is connected to the Internet and the wireless diagnostic module advertising packet includes the Company Vendor Unique Number, Product ID, and Enrollment Status. Once this condition is true, mobile device 18 or router 19 connects to the wireless diagnostic module 12. The mobile device 18 or router 19 may extract the cloud database UID and individual biomedical parameters and push or otherwise transfer all this data to the cloud. Alternatively, the wireless diagnostic module 12 will push or otherwise transfer the data to the mobile device 18 or router 19.

Data may be stored in the cloud database 20 as JavaScript Object Notation (JSON) format, with time stamp header. Other formats may be used.

A user or (mobile device operator) of the client data terminal 14 should be authenticated prior to retrieving any patent information from the cloud database 20. The authentication may also be accomplished through the cloud server 16. This may be done using the same credentials as used for enrolling a new patient.

If a user 24 (doctor or caregiver) is authenticated to view biomedical information for the particular patient 22, de-identified data will be retrieved from the cloud database 20 and transmitted to the client data terminal 14. The biomedical patient data may be presented to the user 24 as tables or graphs. Other visual and auditory representations may be used. The client data terminal 14 may not make any “local copies” of the cloud database 20. In this embodiment, the biomedical patient data is stored in the database 20, while representations of the data may be viewed remote from the patient 22 and remote from the server 16. In the illustrated embodiment, the data received and transmitted by the cloud server 16 is de-identified data with no information about the patient's identity, such that the cloud server 16 can be owned or controlled by an entity different than a hospital, healthcare facility, or other healthcare provider associated with the authorized users of the client data terminals 14. Once the data from the cloud server 16 is provided to the client data terminal 14, the client data terminal 14 can then provide the received data to the medical provider's confidential, secure, protected server. The data can then be correlated to the patient's private, protected records at the medical provider's server to update the particular patient records for current or future use.

The network described herein allows an authenticated user to retrieve de-identified biomedical patient data from the cloud using any computer connected to the internet. Therefore, retrieval of patient data may be accomplished from hardware that is not dedicated to the network 10, enhancing the flexibility of the system.

For a computer, such as the client data terminal 14′, which was discussed previously and may be located remote from the patient, to retrieve data from the cloud-based server 16, the user 22 (doctor or caregiver) signs onto a specific URL and then receives authentication from the cloud-based server 16. If the user 24 is authenticated to view biomedical information for the particular patient 22, data is transmitted from the cloud data-base to the client data terminal 14′, and will may be presented to the user 24 as tables, graphs, and the like.

Furthermore, by using an API in the mobile device 18, connectivity to the wireless diagnostic device 12 can be facilitated without having dedicated hardware which enhances the flexibility of the system. Privacy is maintained by use of the UID, and by transmitting simple values for the sensed parameters, which can effectively reduce the overhead, cost, required bandwidth, and complexity of the network.

Several implementations of the disclosed technology are described above in reference to the figures. The computing devices on which the described technology may be implemented can include one or more central processing units, memory, input devices (e.g., keyboard and pointing devices), output devices (e.g., display devices), storage devices (e.g., disk drives), and network devices (e.g., network interfaces). The memory and storage devices are computer-readable storage media that can store instructions that implement at least portions of the described technology. In addition, the data structures and message structures can be stored or transmitted via a data transmission medium, such as a signal on a communications link. Various communications links can be used, such as the Internet, a local area network, a wide area network, or a point-to-point dial-up connection. Thus, computer-readable media can comprise computer-readable storage media (e.g., “non-transitory” media) and computer-readable transmission media.

Reference in this specification to “embodiments” or “implementations” (e.g., “some implementations,” “various implementations,” “one implementation,” “an implementation,” etc.) means that a particular feature, structure, or characteristic described in connection with the implementation is included in at least one implementation of the disclosure. The appearances of these phrases in various places in the specification are not necessarily all referring to the same implementation, nor are separate or alternative implementations mutually exclusive of other implementations. Moreover, various features are described which may be exhibited by some implementations and not by others. Similarly, various requirements are described which may be requirements for some implementations but not for other implementations.

As used herein, being above a threshold or exceeding a limit means that a value for an item under comparison is above a specified other value, or that the item under comparison is outside of a specified range, that an item under comparison is among a certain specified number of items with the largest value, or that an item under comparison has a value within a specified top percentage value. As used herein, being below a threshold means that a value for an item under comparison is below a specified other value, that an item under comparison is among a certain specified number of items with the smallest value, or that an item under comparison has a value within a specified bottom percentage value. As used herein, being within a threshold means that a value for an item under comparison is between two specified other values, that an item under comparison is among a middle specified number of items, or that an item under comparison has a value within a middle specified percentage range. Relative terms, such as high or unimportant, when not otherwise defined, can be understood as assigning a value and determining how that value compares to an established threshold.

As used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item such as A and A; B, B, and C; A, A, B, C, and C; etc.

Although the subject matter has been described in language specific to structural features and/or methodological acts, it is to be understood that the subject matter defined in the appended claims is not necessarily limited to the specific features or acts described above. Specific embodiments and implementations have been described herein for purposes of illustration, but various modifications can be made without deviating from the scope of the embodiments and implementations. The specific features and acts described above are disclosed as example forms of implementing the claims that follow. Accordingly, the embodiments and implementations are not limited except as by the appended claims.

Any patents, patent applications, and other references noted above are incorporated herein by reference. Aspects can be modified, if necessary, to employ the systems, functions, and concepts of the various references described above to provide yet further implementations. If statements or subject matter in a document incorporated by reference conflicts with statements or subject matter of this application, then this application shall control.

I claim:

1. A system configured to wirelessly transmit biomedical patient data, comprising:
a diagnostic module configured to collect biomedical patient data from a patient and wirelessly transmit a data packet comprising a unique identifier associated with the patient and at least a portion of the biomedical patient data, and wherein the unique identifier and the biomedical patient data in the data packet contain no private personal information about the patient's identity;
a mobile device configured to wirelessly receive the data packet from the diagnostic module and wirelessly transmit the data packet; and
a cloud-based server configured to wirelessly receive the data packet from the mobile device, the cloud-based server has a database and is configured to correlate the patient data via the unique identifier with the patient and to store the data packet in the database, and the cloud-based server is configured to provide some or all of the data packet to an authorized recipient via a client data terminal configured to correlate the unique identifier to patient information at the client data terminal.

2. The system of claim 1, further comprising the client data terminal, wherein the client data terminal is configured to wirelessly receive the data packet from the cloud-based server, the client data terminal comprising an output for presenting information associated with the data packet.

3. The system of claim 1 wherein the diagnostic module is a first diagnostic module, and wherein the collected patient data is first patient data, and further comprising at least a second diagnostic module configured to collect second patient data from the patient and wirelessly transmit to the mobile device the second patient data and the unique identifier.

4. The system of claim 1, further comprising the client data terminal, wherein the client data terminal is configured to wirelessly transmit patient identification data associated with the patient to the cloud-based server, and further comprising a database configured to be in communication with the cloud-based server to generate the unique identifier and to store the unique identifier, the patient identification data, and the data packet in the database.

5. The system of claim 1 wherein the diagnostic module is a blood flow sensor, EKG monitor, pulse oximeter, temperature sensor, movement sensor, or blood pressure sensor, or any combination thereof.

6. The system of claim 1 wherein the data packet further comprises real-time patient data collected over a predetermined amount of time, the diagnostic module being configured to periodically transmit a series of the data packets acquired over time.

7. The system of claim 1, further comprising the client data terminal, wherein the client data terminal is configured to request from the cloud-based server patient data associated with the patient, wherein the cloud-based server is configured to wirelessly transmit the patient data to the client data terminal upon an authentication of the client data terminal.

8. The system of claim 1 wherein the diagnostic module has a module identifier unique to the diagnostic module, and wherein the mobile device wirelessly detects the diagnostic module, the mobile device is configured to associate the wireless diagnostic module with the server based on the unique identifier and the module identifier.

9. The system of claim 1 wherein the data packet is a first data packet, and the diagnostic module can transmit a second data packet different than the first data packet, wherein the mobile device is configured to ignore the second data packet when the second data the unique identifier is not correlated to the patient.

10. The system of claim 1 wherein the mobile device is configured to store a series of the data packets, the mobile device is configured to transmit one file to the cloud-based server comprising the series of data packets.

11. The system of claim 1 wherein the mobile device is configured to authenticate the diagnostic module based on at least one of the unique identifier and unique module identifier associated with the diagnostic module.

12. The system of claim 1 wherein the diagnostic module is a Released Device or an Enrolled Device.

13. A method for wirelessly collecting and saving biomedical patient data, comprising:
defining a de-personalized unique identifier for a patient by receiving, at a server, patient identification data transmitted from a client data terminal, and generating a unique identifier associated with the patient identification data at the server, wherein the patient identification data includes private personal information about the patient and the patient's identity, and wherein the unique identifier contains no private personal information about the patient and the patient's identity;
associating a diagnostic module with the unique identifier;
providing the unique identifier from the server to the diagnostic module, wherein the diagnostic module is configured to collect biomedical patient data from a patient and to wirelessly transmit a data packet comprising the unique identifier and at least a portion of the real-time biomedical patient data, and wherein the data packet contains no private personal information about the patient's identity; and
receiving, at the server, the data packet sent from the diagnostic module.

14. The method of claim 13, further comprising saving at least a portion of the data packet, including the unique identifier, in memory accessible by the cloud-based server.

15. The method of claim 13, further comprising wirelessly providing the at least a portion of the data packet from the server to a remote client data terminal authorized to receive the biomedical patient data in the at least a portion of the data packer:

16. The method of claim 15, further comprising:
receiving, at the server, an alarm indicating that the biomedical patient data has exceeded a limit; and
transmitting to the client data terminal an alert associated with the unique identifier and the alarm.

17. The method of claim 13 wherein the receiving the data packet further comprises receiving, at the server, the data packet from a mobile device or a data router that receives the data packet from the diagnostic module.

18. A method for wirelessly transmitting biomedical patient data, comprising:
receiving at a cloud-based server a data packet comprising a unique identifier associated with a patient and at least a portion of biomedical patient data collected in real time from the patient, wherein the unique identifier includes no private personal information about the patient or the patient's identity, wherein the data packet is wirelessly received from a mobile device or a router that has received the portion of the biomedical patient data and the unique identifier associated with the patient from a diagnostic module;
authenticating a client data terminal with the cloud-based server; and
wirelessly transmitting, from the cloud-based server to the client data terminal, the data comprising the unique identifier and at least a portion of the biomedical patient data, wherein the data wirelessly transmitted to the client data terminal contains no private personal information about the patient's identity.

19. The method of claim 18, further comprising:
receiving, at the cloud-based server, an alarm indicating that the biomedical patient data has exceeded a limit; and
transmitting to the client data terminal an alert associated with the unique identifier and the alarm.

20. The method of claim 18 wherein the diagnostic module is a first diagnostic module and the data packet is a first data packet, the method further comprising:
receiving at the cloud-based server a second data packet comprising the unique identifier associated with the patient and at least a portion of second biomedical patient data collected in real time from the patient, wherein the second data packet is wirelessly received from the mobile device or the router that has received the portion of the second biomedical patient data and the unique identifier associated with the patient from a second diagnostic module; and
wirelessly transmitting from the cloud-based server to the client data terminal second data comprising the unique identifier and at least a portion of the second biomedical patient data, wherein the second data wirelessly transmitted to the client data terminal contains no private personal information about the patient's identity.
